(19) United States
(12) Patent Application Publication
(10) Pub. No.: US 2015/0148840 A1
(43) Pub. Date: May 28, 2015

(54) TWEEZERS

(71) Applicant: Richard Walter Francis, Bend, OR (US)

(72) Inventor: Richard Walter Francis, Bend, OR (US)

(21) Appl. No.: 14/088,105

(22) Filed: Nov. 22, 2013

(51) Int. Cl. A61B 17/30

(52) U.S. Cl. CPC A61B 17/30
USPC 606/210

(57) ABSTRACT

Tweezers are disclosed for example folding tweezers. In some implementations the tweezers include a protective member and a tweezer member pivotably mounted on the protective member to allow the tweezer member to rotate relative to the protective member between a first position, in which the tweezer member is protected by the protective member, and a second position, in which the tweezer member is exposed. The tweezer member may be mounted adjacent the distal end of the protective member, and may include a portion configured to extend beyond the distal end of the protective member when the inner tweezer member is in the first position.

### BACKGROUND

Tweezers are a well-known, simple mechanical device that is produced in many different forms to fulfill various needs such as hair removal, wound debridement and generally manipulation of small things.

### SUMMARY

In general, various types of tweezers, and in particular folding tweezers, are disclosed herein.

In one aspect, the invention features a device comprising a protective member and a tweezer member pivotably mounted on the protective member to allow the tweezer member to rotate relative to the protective member between a first position, in which the tweezer member is protected by the protective member, and a second position, in which the tweezer member is exposed. The tweezer member is mounted adjacent the distal end of the protective member, and includes a portion configured to assist a user in moving the tweezer member from the first position to the second position.

Some implementations may include one or more of the following features. The portion may extend beyond the distal end of the protective member when the inner tweezer member is in the first position.

The device may be configured so that the portion of the tweezer member acts as a lever to facilitate movement of the tweezer member to the second position. The protective member may be an outer member defining an opening to receive the tweezer member. In some cases, the lever portion of the tweezer member may extend at least 3 mm, e.g., from 3 to 25 mm, from the distal end of the protective member. In some implementations, the lever portion may extend beyond the mounting point of the tweezer member on the protective member,

The tweezer member may be mounted on the protective member by engagement of corresponding male and female elements, e.g., by protrusions that extend from the protective member into recesses in the tweezer member or vice versa.

In some cases, the protective member may be substantially U shaped.

In some implementations, the tweezer member and the protective member include corresponding detents and recesses that are configured to releasably arrest the rotation of the tweezer member with respect to the protective member. In some cases, the detents may be oriented to releasably hold the tweezer member in an open and alternatively closed position with respect to the protective member.

In another aspect, the invention features a device comprising a protective member and a tweezer member having a pair of opposed tip portions. The tweezer member is pivotably mounted on the protective member to allow the tweezer member to rotate relative to the protective member between a first position, in which the tweezer member is protected by the protective member, and a second position, in which the tweezer member is exposed. In this aspect, at least a portion of the tweezer member is formed of plastic, and the tip portions are formed of a metal.

Some implementations of the invention may feature one or more of the following features. In some cases, at least a portion of the protective member may be plastic. In some cases, the entire protective member may be constructed of plastic. In some implementations, the plastic may be injected molded.

In some cases, the tips may snap fit into a plastic portion of the tweezer member. In other implementations, the tips may be co-molded into the plastic portion of tweezer member.

In another implementation, the entire tweezer member may be constructed of a metal or metal alloy.

In another aspect, the invention features a device comprising a protective member and a tweezer member having a pair of opposed tip portions, the tweezer member being pivotably mounted on the protective member to allow the tweezer member to rotate relative to the protective member between a first position, in which the tweezer member is protected by the protective member, and a second position, in which the tweezer member is exposed, wherein at least a portion of the protective member is formed of plastic, and the protective member is configured to surround the tip portions of the tweezer member.

In some cases, the entire tweezer member may be constructed of metal.

In another aspect, the invention features a device comprising a tweezer member having a pair of elongated arms that are joined at a proximal end, and that each extend at a distal end a tip portion. Each tip portion has a generally planar anvil region, the anvil regions configured to contact each other when the arms are urged toward each other by a user, and a pointed tip, the tip having a first tip angle, measured in the plane of the two arms, of from 15 degrees to 25 degrees, and a second tip angle, measured in the plane of the anvil portion, of from 15 degrees to 37 degrees.

In some implementations, the pointed tip has a radius of curvature of less than 0.127 mm. The anvil region preferably extends at least 2 mm, e.g., at least 4 mm, from the pointed tip towards the proximal end of the arm. The device is preferably configured such that the pointed tips will contact each other at the same time or before the anvil regions contact each other. The device may further include an outer, protective member on which the tweezer member is mounted, and may include any of the other features described herein.

### DESCRIPTION OF THE DRAWINGS

FIG. 1 is a perspective view of a device according to one embodiment in a closed position

FIG. 2 is a perspective view of the device of FIG. 1 in an open position.

FIG. 3 is a perspective view of the protective member shown in FIG. 1.

FIG. 4 is another perspective view of the protective member shown in FIG. 1.

FIG. 5 is a perspective view of the inner member shown in FIG. 1.

FIG. 6 is a perspective view of an alternative inner member that may be used with the outer member shown in FIG. 4.

FIG. 6A is a top view of the inner member shown in FIG. 6.

FIG. 6B is a side view of the inner member shown in FIG. 6.

FIG. 6C is a transverse view of the inner member shown in FIG. 6A shown from the top.

FIG. 7 is a perspective view of a third embodiment of the inner member.

FIG. 7A is a cross-sectional view taken along line A-A in FIG. 7.

FIG. 8 is a perspective view of a fourth embodiment of the inner member.

### DETAILED DESCRIPTION

The present disclosure relates generally to tweezers designed to fold for easy and safe transport. Tweezers are a hand-held instrument designed to perform a task such as grasping or prying objects that are too small to be manipulated by the fingers. Tweezers may be used, for example to remove splinters or ticks. The preferred embodiment of the device is designed, when closed, to easily fit in a purse, pocket, or hang from a key ring. As will be discussed below, in preferred embodiments the tweezers include sharp, pointed tips. These tips are protected from undesirable contact with the user when the device is closed.

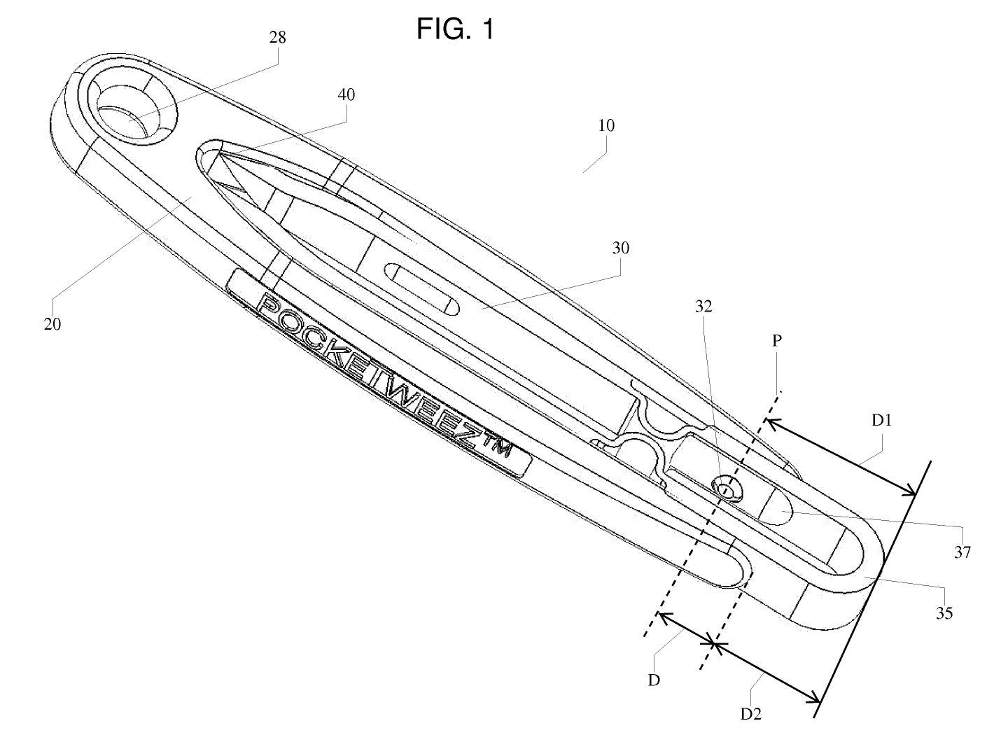

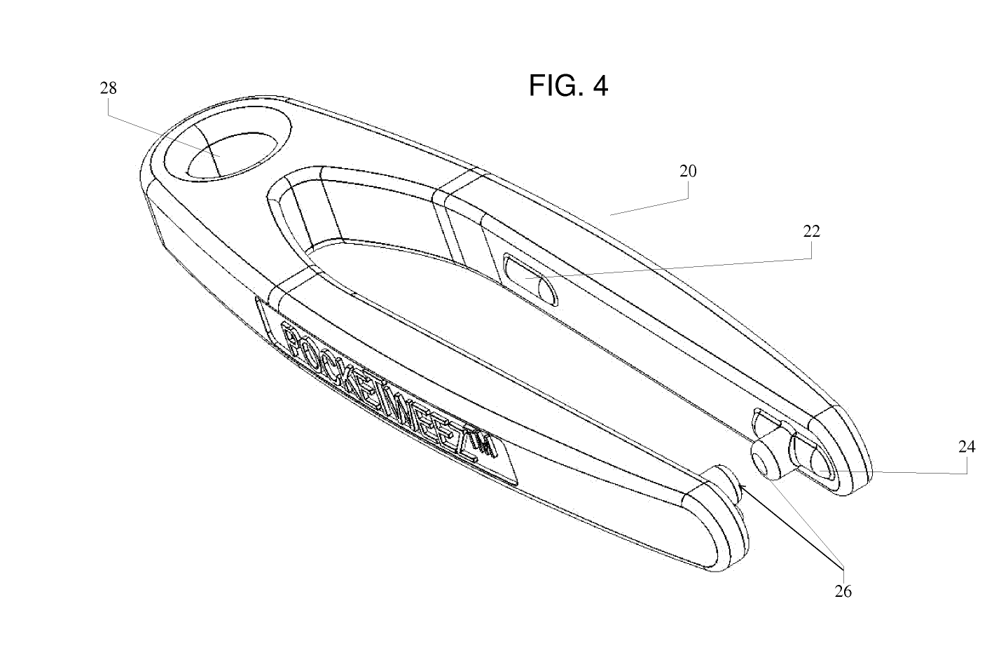

Referring to FIG. 1, the device 10 has an outer, protective member 20 and an inner, tweezer member 30. The tweezer member 30 is dimensioned to be received by the protective member 20 when in a closed position as shown in FIG. 1. When in the closed position, tips 40 are fully protected by protective member 20. The inner member rotates on a set of pivot pins 26 (FIG. 4) that are received in cooperating recesses located adjacent the distal end of the protective member 20. For example, the distance D between the pivot axis P and the distal end of the outer member may be about 5 mm, for example, from about 1 to 10 mm.

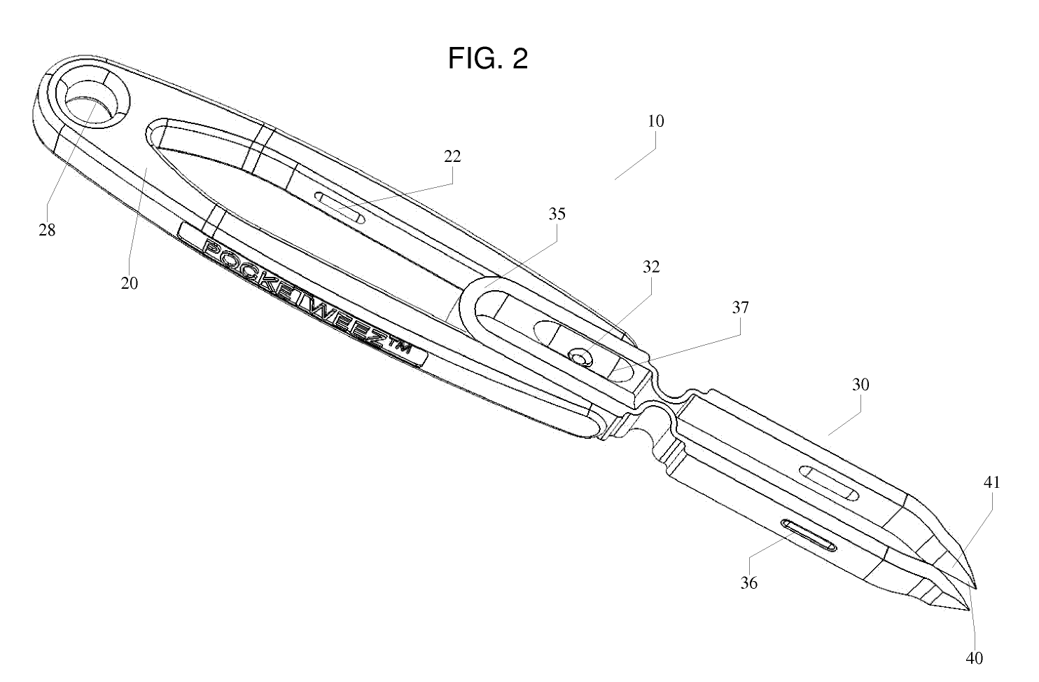

A lever portion 35 extends beyond the distal end of the protective member 20. The lever portion 35 offers a mechanical advantage to the user when the device is rotated from the closed position (FIG. 1) to the open position (FIG. 2). The lever portion 35 allows the user to more easily rotate the device 10 into the open position where the tips 40 are exposed and ready for use. In some implementations, the end of lever 35 is at least about 13 mm from the pivot axis P, e.g., from 9 to 20 mm (D1), and about 7 mm, e.g. 3 to 15 mm, from the distal end of the outer member (D2).

Hole 28 serves as an attachment point where the user can attach the device 10 to a ring or lanyard, e.g. a key ring. Preferably all the edges on the protective member 20 and the tweezer member 30, except for the tips 40, are rounded, or radiused, to reduce damage to any storage container, for example a pocket or purse.

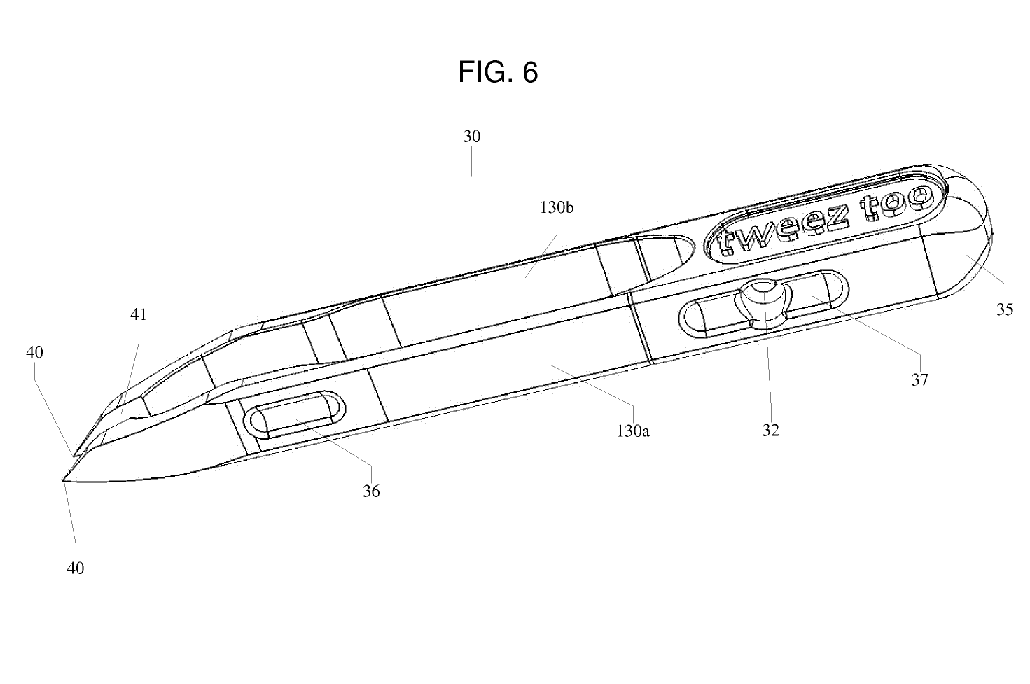

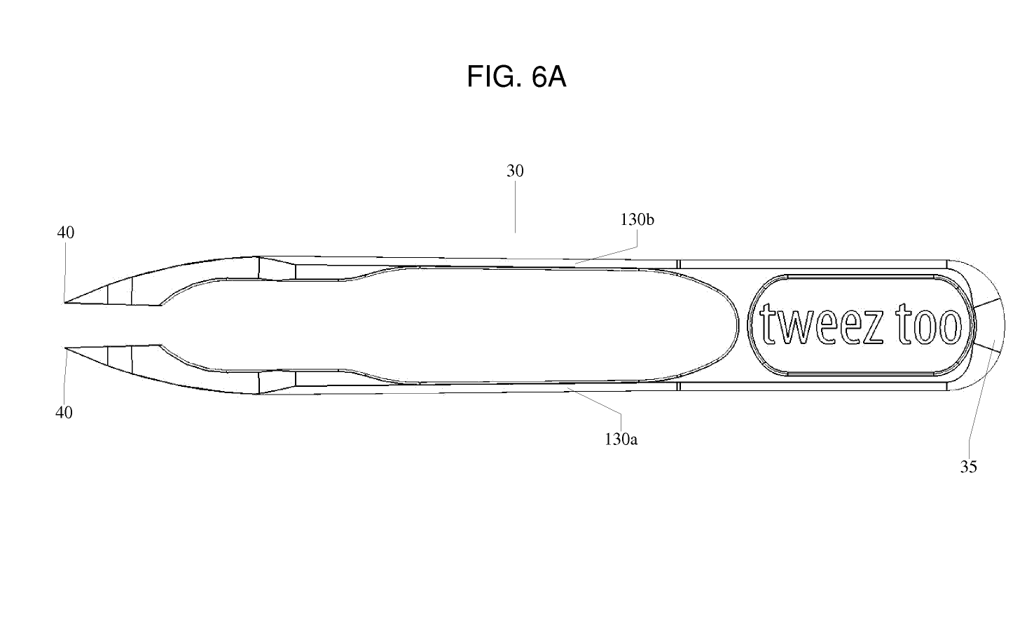

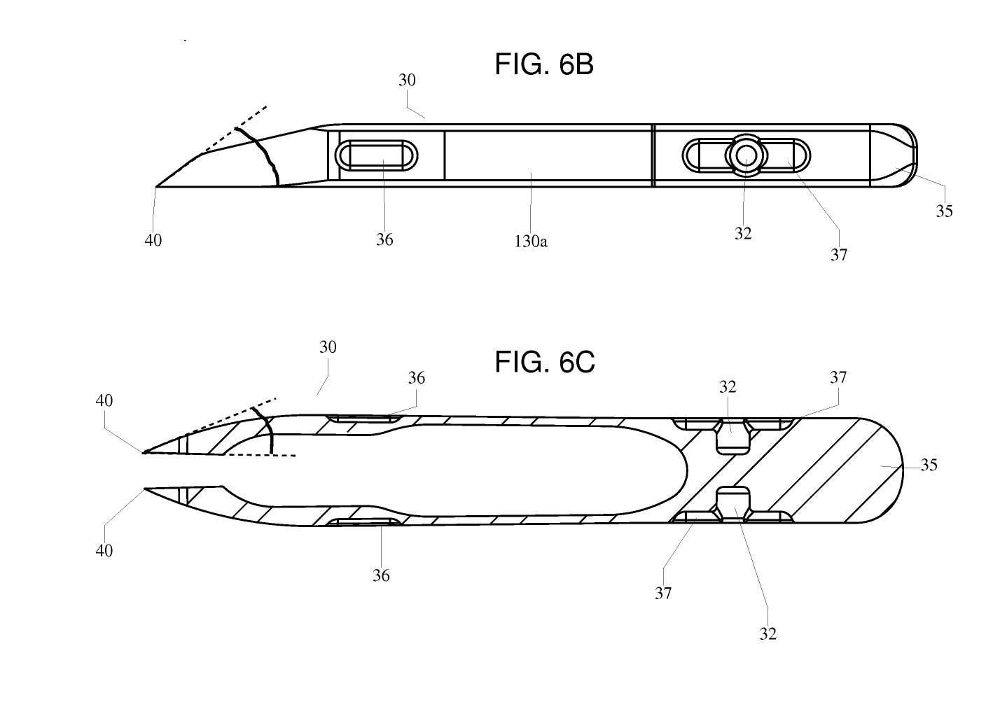

Referring to FIG. 2, the device 10 is shown in the open position where the tips 40 are available for use. The tips 40 are generally shaped/angled to enable the user to successfully grasp, pry, or generally manipulate small objects, e.g. slivers, insects, thorns etc. Preferably, the tip angle as measured in the plane of the arms 130a, 130b of the tweezer member (FIG. 6B) is from about 15° to 25°. The tip angle as measured in the plane of anvil 41 (FIG. 2) is about 37°, e.g., from about 15° to 37°. In some implementations the pointed tip has a radius of curvature of less than 0.127 mm. Preferably, the tip region begins to taper a minimum of 15 mm from the tip to aid visibility of grasping a target. Anvil 41 is a flat surface located on the inner aspect of the tips 40. Preferably, anvil 41 is at least 2 mm in length (measured from tip to terminus), e.g. 1 mm to 6 mm. During use, e.g. when grasping a small object, the tweezer member is configured so that the pointed tips will come into contact before or at the same time as the anvils to ensure secure capture.

When the device 10 is in an open position, tips 40 are available for use. When the device 10 is in the closed position, the body of the protective member 20 protects tips 40. The tweezer member 30 is configured to flex in a plane that is substantially parallel to the axis of rotation allowing tips 40 to come in contact with one another. In the preferred embodiment, the force required to bring the tips 40 into contact is 8.896 N to 9.786 N or 4.448 N to 4.893 N per tweezer leg 130a, 130b (FIG. 6). The force is affected by a combination of the thickness of the tweezer legs 130a, 130b (FIG. 6) and the material used for construction. If the material used is relatively stiff, e.g. Stainless Steel, the central portion of the tweezer legs 130a, 130b (FIG. 6C) is thin, e.g., less than 1.25 mm and preferably less than 1 mm, e.g., about 0.64 mm.

Referring to FIGS. 1, 2, 4, and 6, a pair detents 22 (FIG. 2) are positioned on the inner surface 21 of protective member 20 to be received in recesses 36 of the inner member and thereby mechanically resist free rotation of the inner member. This engagement helps to keep in the inner member in the closed position (FIG. 1) when the tweezers are not in use. Similarly, an additional pair of detents 24 and recesses 37 is provided adjacent at the distal end of protective member 20 and proximal end of tweezer member 30. Detents 24 help maintain the tweezer member 30 in a closed position and also secure the tweezer member in place when in the open position (FIG. 2). The detents, 22, 24 also help center the tweezer member 30 with respect to the protective member 20. Fillets surrounding recesses 36 and 37 allow for smoother engagement and disengagement of the detents.

To open the device, a user would grasp the protective member 20 with the thumb, first and second fingers of one hand and then either push or pull on lever portion 35. Once sufficient force is generated to overcome detents 22, 24, the tweezer member 30 rotates about an axis established by pivot pins 26, generally 180°, until detents 24 are again received by recesses 37, locking the tweezer member 30 into the open position (FIG. 2).

The pivot pins 26 and the corresponding pivot bore 32 are designed to allow smooth rotation, while preventing unintended disassembly of the tweezer member 30 from the protective member 20. The preferred diameter of the pivot pins 26 is 2.5 mm but can range from about 1 mm to 4 mm. The length of pivot pins 26 can range from about 0.75 mm to 3.5 mm, e.g., from 1.5 to 2.0 mm. The pivot pins 26 and pivot bore 32 are configured such that the tolerance allows for rotation about the intended axis while minimizing unwanted lateral movement. Preferably the pivot bores 32 are 0.0254 mm-0.254 mm larger in diameter than the pivot pins 24. The depth of the pivot bore 32 is sufficient to accept the full length of the pivot pins without bottoming out.

Advantageously, the device is easy to open as a result of a combination of the physical characteristics of the materials used in construction and the design of the detents and recesses. In some embodiments, the force required to open the device is less than about 9.786 N preferably less than 8.896 N and in some cases even less than 6.672 N. For example, the force could be between 0.889 N and 9.786 N. The force is tested by clamping the end of the handle with hole 28 to a horizontal shelf so the rest of the assembly is cantilevered over the edge. An adjustable weight is hung from the tweezer at a distance 23.75 mm from the pivot point. Weight is added in 43 gram increments until the tweezer member 30 disengages from the protective member 20.

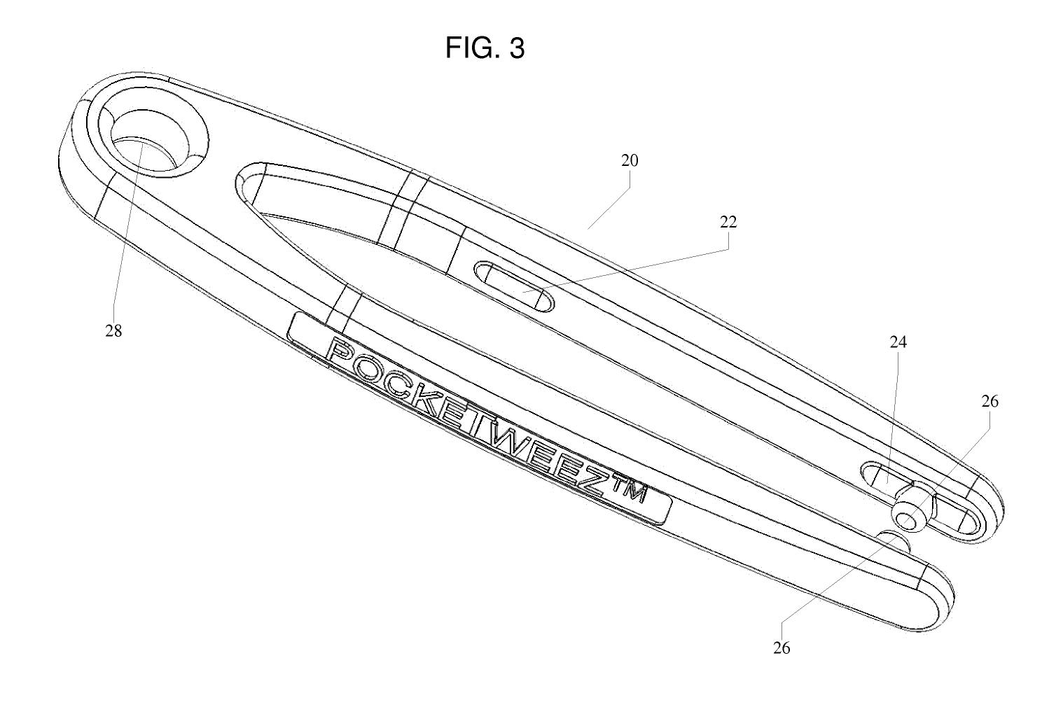

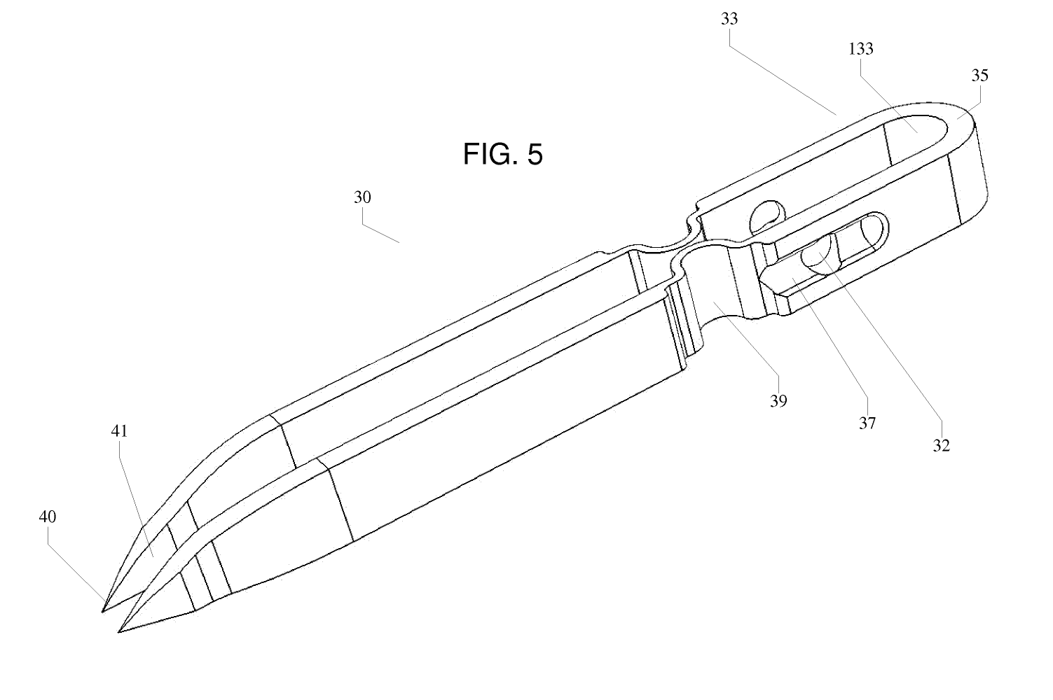

Referring to FIG. 5, in the embodiment shown in FIGS. 1-5 the proximal portion 33 of the tweezer member 30 defines a central opening 133. A necked-in region 39 is provided in this embodiment so that flexing the legs 130a, 130b of tweezer member 30 towards each other will not disengage pivot pins 26 from pivot bore 32. This feature is not necessary in the embodiments shown in FIGS. 6-8, in which the proximal portion of the tweezer is solid.

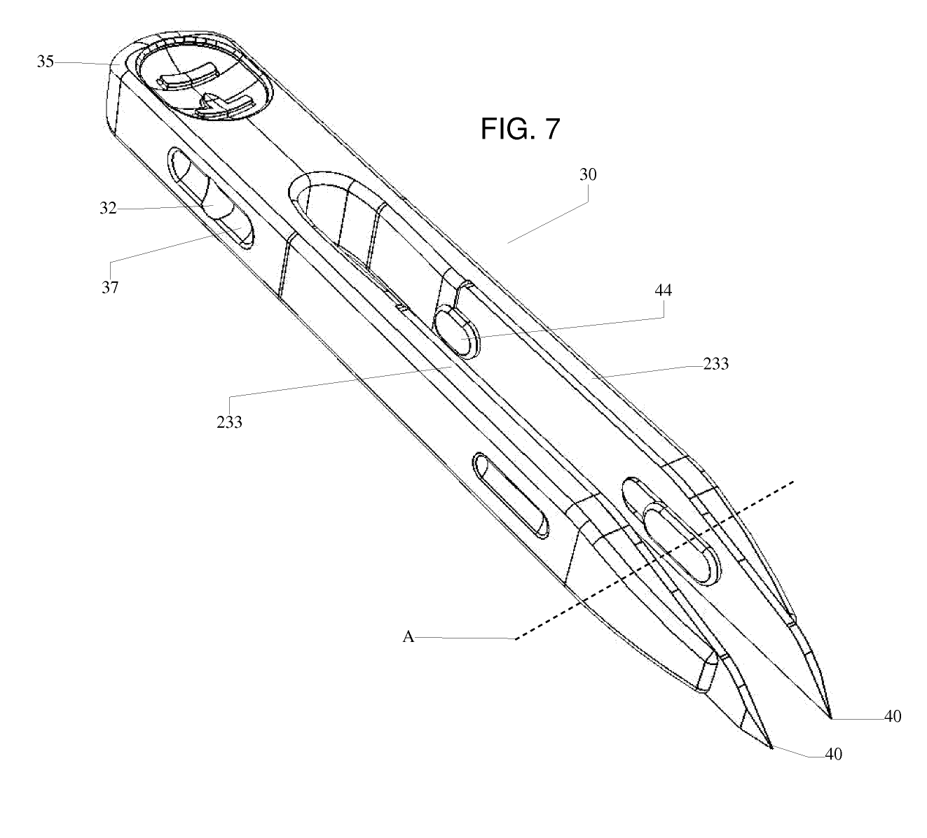

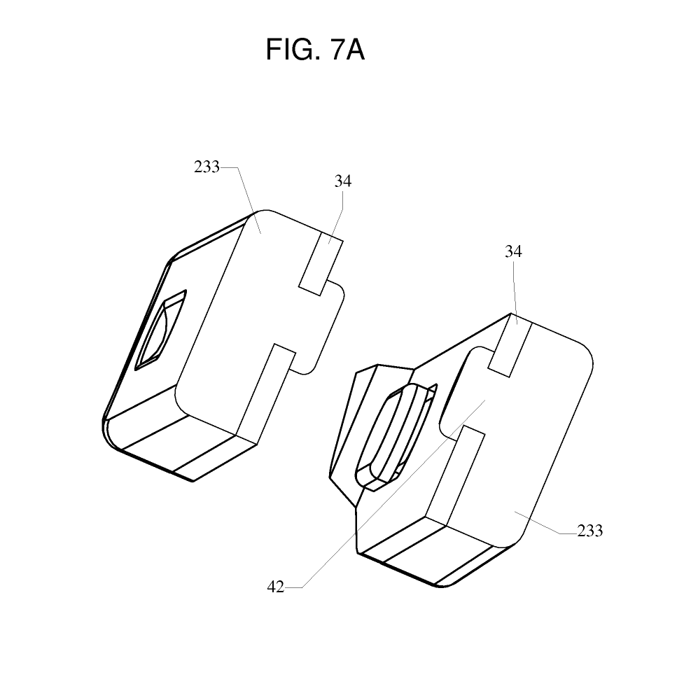

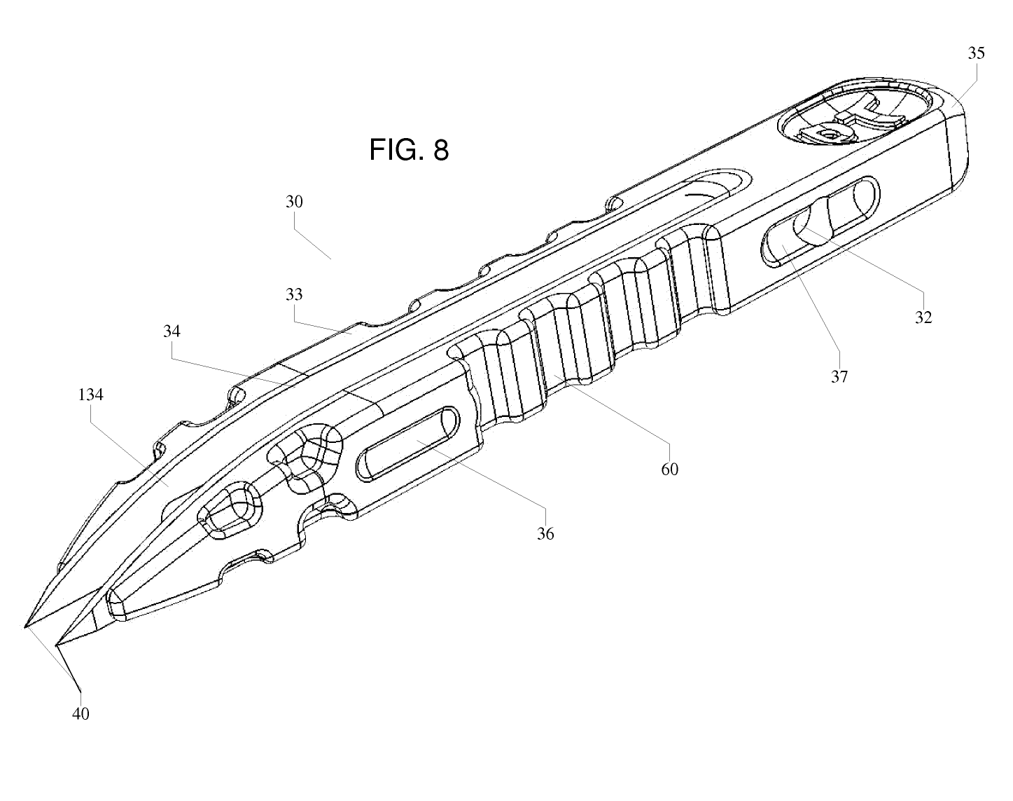

FIGS. 7 and 7A show a partial plastic tweezer member 33 that includes plastic legs 233 with metal alloy tip inserts 34. Metal alloy tip inserts 34 can be snap-fit to plastic tweezer member 30. This configuration would allow easier manufacturing, enable tip replacement due to wear or damage, or enable the user to quickly adapt the tweezer for an alternative type of manipulation, e.g. a fine point tip as opposed to a broad, round-nosed tip. For example, as shown in FIG. 7A, tip inserts 34 can include an opening (not shown) configured to snap over a radiused protrusion 42. Protrusion 42 includes a stem 41, configured to fit with the opening, and a button 43, configured to overhang the opening, providing an interference fit and retaining the tip 34 in place. Plastic tweezer member 33 can be molded to the metal alloy tip inserts 34, e.g. using insert molding. The metal insert can be just the tips (FIG. 7), or a substantially U-shaped piece 134 (FIG. 8) that fits on the inner surface of the tweezer member.

As shown in FIG. 8, contours 60 may be formed in the tweezer member 33 to increase torsional rigidity and enhance the spring characteristics. The contours may also increase the resistance to lateral deflection of the tweezer member during tweezing. Contours 60 also provide a positive gripping surface.

The device shown in FIGS. 1-5, as well as the alternative tweezer members shown in FIGS. 6-8, may be made of any suitable materials that enable effective use of the tweezer member and that prevent unintended disengagement of the tweezer member from the protective member.

In some cases, the device includes a combination of different materials. For example, the protective member may be formed of plastic while the tweezer member is formed partially or entirely of metal. Alternatively, the entire device may be formed of metal.

Metal portions of the device may be made using metal injections molding (MIM), which facilitates production of the sharp tips of the tweezer member and which is generally less expensive and faster than machining However, if desired the parts may be made using machining or other metalworking processes.

The metal used is preferably corrosion and chemical resistant, and, for the tweezer member, has appropriate spring characteristics for use in tweezers. Suitable metals include 17-4 PH Stainless Steel and other 400 Series Martensitic Stainless Steels hardened to a target hardness of about Rockwell C30 to Rockwell C45. Preferably the materials used have a yield strength that prevents them from permanently deforming during normal use of the device.

Many rigid thermoplastics may be used for the protective member, e.g., nylons, polypropylene, polyethylene, etc. When plastic is used for the tweezer member, it is important that the plastic have suitable flexural characteristics, and, if used to form the tips, preferably have sufficient durability to maintain tip sharpness. Suitable plastics include those used to for precision anti-static tweezers, e.g., glass-filled acetal polymers such as DELRIN® and CELCON® polymers.

### OTHER EMBODIMENTS

A number of embodiments have been described. Nevertheless, it will be understood that various modifications may be made without departing from the spirit and scope of the disclosure.

In some implementations, the device may be sized differently, either larger or smaller, in which case the dimensions would generally remain proportional.

Another embodiment of the device may feature textured tips. The texture would enhance the gripping ability of the tweezers. Moreover, while sharp tips are preferred for many applications, in some implementations the tips may be blunt.

Some implementations of the device may feature alternate locations of the detents, fillets and pivot pins. For example, the detents or fillets may be configured on the inner member, the outer member or any combination thereof. Moreover the fillets could be replaced by chamfers if desired. Also the size of the detents, recesses, the pivot pins and pivot bores may vary depending on the materials uses and are selected to provide the desired opening force and resistance to inadvertent disengagement of the pivot pins from the pivot bores.

Another embodiment of the device may feature only the tweezer member without the protective member. For example, the tip geometry described herein may be used in tweezers other than folding tweezers.

In addition, while a lever portion, extending beyond the pivot point of the tweezers, is described above, other features may be included instead or in addition to the lever to assist the user in opening the tweezers. For example, a raised feature may be included on the tweezer member near the pivot point to give the user something to grasp or push against. In some implementations the lever may be omitted and no feature provided to facilitate opening of the tweezers.

Accordingly, other embodiments are within the scope of the following claims.

1. A device comprising:
a protective member comprising a pair of elongated arms terminating at distal ends; and
a tweezer member comprising a pair of elongated arms terminating in tweezer tips, the tweezer member being pivotably mounted on the protective member to allow the tweezer member to rotate relative to the protective member between a first position, in which the tweezer tips are protected by the protective member, and a second position, in which the tweezer tips are exposed,
wherein the tweezer member is mounted at a mounting location adjacent the distal ends of the protective member, and includes a lever portion that extends beyond the distal ends of the protective member when the tweezer member is in the first position and acts as a lever to assist a user in moving the tweezer member from the first position to the second position; and
wherein, when the tweezer member is in the first position, the elongated arms of the tweezer member are disposed adjacent to the elongated arms of the protective member, defining an open central area.

2-3. (canceled)

4. The device of claim 1, wherein the protective member is an outer member defining an opening to receive the tweezer member.

5. The device of claim 1, wherein the lever portion of the tweezer member extends at least 3 mm from the distal ends of the protective member.

6. The device of claim 1, wherein the lever portion of the tweezer member extends from about 3 to 25 mm, from the distal ends of the protective member.

7. The device of claim 1, wherein the lever portion extends beyond the mounting location of the tweezer member on the protective member a distance of at least about 13 mm.

8. The device of claim 1, wherein the tweezer member is mounted on the protective member by engagement of corresponding male and female elements.

9. The device of claim 1, wherein the protective member is substantially U shaped.

10. The device of claim 1, wherein the tweezer member and the protective member include corresponding detents and recesses configured to releasably arrest the rotation of the tweezer member with respect to the protective member.

11. The device of claim 10, wherein the detents and recesses are oriented to releasably hold the tweezer member in the first position and the second position with respect to the protective member.

12-18. (canceled)

19. The device of claim 1 wherein at least a portion of the protective member is formed of plastic, and the protective member is configured to surround the pointed tips of the tweezer member.

20. The device of claim 19, wherein the entire tweezer member is constructed of metal.

21. The device of claim 1 wherein the lever portion extends at least about 3 mm beyond the distal ends of the protective member.

22. The device of claim 1 wherein the tweezer tips are pointed.

23. The device of claim 22 wherein the tweezer tips have a tip angle of about 15 to 25 degrees, measured in a plane indicated in FIG. 6B.

24. The device of claim 22 wherein the tweezer tips have an anvil tip angle of about 15 to 37 degrees, measured in a plane indicated in FIG. 6C.

25. The device of claim 10 wherein the detents and recesses are disposed on the adjacent arms of the tweezer member and protective member.
